(19) United States
(12) Patent Application Publication
(10) Pub. No.: US 2003/0044771 A1
(43) Pub. Date: Mar. 6, 2003

(54) Method for discovering new infectious particles

(76) Inventors: Norman G. Anderson, Rockville, MD (US); N. Leigh Anderson, Washington, DC (US); Guy Della-Cioppa, Vacaville, CA (US)

Correspondence Address: Dean H. Nakamura, Roylance Abrams Berdo & Goodman, 1300 19th Street, N.W., Washington, DC, 20036, US

(21) Appl. No.: 09/941,626

(22) Filed: Aug. 30, 2001

(51) Int. Cl. C12Q 1/70; C12Q 1/68; G06F 19/00; G01N 33/48; C12P 19/34; G01N 33/50

(52) U.S. Cl. 435/5; 435/6; 435/91.2; 702/20

(57) ABSTRACT

The isolation and characterization of multiple viruses from a sample is provided.

### FIELD OF THE INVENTION

The present invention relates the isolation and characterization of multiple viruses from a mixed biological sample.

### BACKGROUND OF THE INVENTION

While scientists have been isolating new microorganisms for over 100 years, new viruses and variants of existing viruses are continuing to be found. Further, experimental and epidemiological evidence supports the view that a large number of infectious agents remain to be discovered. In addition, new variants of known ones constantly occur. In many instances, the putative agents have not been demonstrated because they cannot be cultured in vitro using available techniques. Diseases postulated to be due to undiscovered agents include schizophrenia, diabetes, atherosclerosis, multiple sclerosis, leukemia and others. In the first phase of the CDC study of Unexplained Deaths due to Possible Infectious Causes (UDPIC), deaths were monitored by the CDC Emerging Infections Program (EIP), and 13% of hospitalized deaths among persons 1-49 years old who were previously healthy were classified in this category. Much higher rates were observed in older individuals. Every year, 3-5 previously healthy individuals per 100,000 population die with symptoms of an infectious disease but without a confirmed diagnosis despite use of state-of-the-art diagnostic technology, [Perkins et al, Emerging Infectious Diseases 2(1): 47-53 (1996)]. Most of these are thought to be viral diseases because they did not response to antibiotics.

The classical techniques for the positive diagnosis of an infectious agent include amplification by in vitro culture, identification based on cell types used for culturing (for viruses), culture conditions, growth inhibition by specific antibodies, detection with labeled antibody or labeled nucleic acid probes, use of the polymerase chain reaction to amplify bacterial or viral DNA or cDNA, or by demonstrating the presence of specific convalescent antibodies.

Conventional viral discovery has started with a disease or suspected disease, and biological samples are cultured and destruction of the culture (cytopathic effect or CPE) is noted. When convalescent serum, presumably containing antibodies to the virus, is available, inhibition of cytopathogenicity is diagnostic. Physical isolation of viruses from a diseased sample followed by culturing has also been attempted. These techniques are readily frustrated when the virus does not grow in various conventional culture media. Conventional virus discovery is difficult, time-consuming and far from certain. Even with great resources devoted to the matter, many years (and deaths) passed before HIV was discovered by these classical techniques.

Using cloning techniques fractional non-host nucleic acid sequences in tissue or serum from individuals infected with a previously uncharacterized agent have been found, and assembled in order to reconstitute a partial or complete viral genome. This was done with human hepatitis C, and the clones and data used to produce antigens that have allowed clinical tests for this agent to be developed without having ever physically isolated or grown the virus. This procedure requires knowledge that a sample contains the virus, is expensive and time-consuming, and cannot be applied routinely to search for new infectious agents.

Certain microorganisms are not culturable even though some are well characterized. One of the most common viruses that infect almost everyone in their lifetime is Norwalk virus. Since it was never cultured in-vitro, human volunteers have been used to produce sufficient quantities for characterization. It required years of effort and unpleasant effects on human volunteers to identify a +sense RNA non-encapsulated 7642 bp virus producing only three proteins. Other “Norwalk-like” calciviruses have also been proposed but have not been identified in cultures. It is evident that for new pathogens, specific antibodies, nucleic acid probes or PCR primers will not be available.

Attempts have been made to search through tissue sections using the electron microscope to find new viruses. Unfortunately at the magnifications required to see viruses in sections, up to 100 8×10 inch micrographs are required to resolve a volume equivalent to a single liver cell. Using electron microscopy putative virions have been described. However, many structures seen in the electron microscope resemble virions, are either not viruses, or are unculturable under presently available laboratory conditions. Simple visualization of an apparent virus does not therefore establish the presence of a true infectious agent and, in any event, cannot determine whether the virus is new or previously known. All of these techniques require much time and effort and are not useful either for rapid diagnosis or for large-scale screening.

Hepatitis delta virus is a natural subviral satellite of human hepatitis B virus (HBV), and can only replicate in or is transmitted in the presence of HBV. Experimentally, the discovery of satellite viruses is impossible in the absence of the virus on which they are dependent. There are believed to exist many viruses, helper viruses, satellite viruses and viroids that escape discovery because they are difficult or impossible to grow in available culture systems.

Historically, viral contaminated articles have been used in warfare including tossing smallpox scabs into enemy camps during siege, distribution of measles contaminated blankets to Native Americans and contamination of water supplies with feces or dead animals. While only a few instances of intentional culturing and distribution of contagious biological warfare agents have actually occurred (Japanese army, Manchuria late 1930's-early 1940's; Soviet Army, Stalingrad, 1943; Rajneeshee cult, Oregon 1984; Aum Shimrikyo cult, Japan, 1990-1995), many threats have been made and the wide availability of biotechnology has raised questions about the future. Also, biological warfare agents directed against crops (rice blunt, wheat and rye stem rust) and livestock (foot and mouth disease) are threats. Criminal activity (extortion, assault, murder, vandalism etc.) with such agents involves essentially the same activity.—Stockpiles of biological warfare agents exist in many locations around the world along with means for their intentional distribution. Given the masses of BW agents that exist, accidental release is always a possibility. Additionally, civilian research and medical labs harbor pathogens that may also be accidentally transmitted.

While much effort has been given to the isolation and characterization of new human viruses, animal viruses are less known and plant viruses are even less studied by comparison. Plant viruses are known to be of great economic harm when virulent, but may have even greater economic harm when not virulent. Viral infected strawberries are well known for degenerating and reducing fruit yield. Grapevine leaf roll virus, citrus tristesa virus, potato virus X and Y, plum pox virus, papaya ringspot virus, tobacco vein mottling virus, sweet potato feathery mottle virus, many mosaic viruses (alfalfa, bean common, beet, johnson grass, maize dwarf, peanut, sorghrum, sugarcane, tobacco, watermelon, wheat streak, yam, zucchini yellow, etc.) all adversely affect plant growth and crop production. Many of these viruses infect multiple food crops and are passed on to subsequent generations in. Insects and other vectors also carry some.

An industry has arisen to provide “virus-free” germ stock. Virus-free designation is currently determined by infectivity tests or immunoassay. However, many previously unrecognized viruses might be present in such material. Infectivity tests are labor intensive, time consuming and prone to error from both false positives and false negatives. Immunoassays can only be done for previously characterized viruses. Viroids are thought to occur chiefly in plants, may occur in animals and man, and are difficult to isolate and characterize.

The natural environment is filled with viruses. The average concentration of viral particles in ocean water, for example, is estimated to be above 10⁵ virions per ml. Given an ocean volume of 1.3×10²⁴ ml, and an average viral mass of 2×10⁻¹⁶ grams, the viral oceanic viral load would be 26 million metric tons, or one third the estimated mass of mankind. Given that a large fraction of oceanic viruses turn over daily and would therefore have a high mutation rate, marine viruses constitute the largest source of new nucleic acid sequences on earth. It has been proposed that all cellular organisms can be, or are infected with viruses, and that viruses transmit genetic information across species and even phylum barriers (Anderson, 1972). Given an estimated over two million species of plants and animals on earth, it would appear that not only a very large number of virus species and variants exist, but that they constitute a large fraction of the biosphere. Since the average individual has over two viral infections per year, with over 140 estimated per lifetime, viruses not only major constituents of our environment, but the cause of most human illnesses.

In the past, virus discovery and characterization has been a “one-at-a-time” effort. Given the present threat of bioterrorism, the large number of pathogenic viruses already characterized, and the conclusion that large numbers of them remain to be discovered, there is now an urgent need for an integrated technology for detecting, isolating and identifying new infectious agents from a variety of different sources, without hazard to operating personnel, that can be applied to samples that may contain more than one infectious agent, and that can characterize large numbers of known and unknown viruses simultaneously. Such systems and methods should be applicable to viruses as a class, and not depend on the class or type of virus.

The highest resolution physical virus isolation techniques previously described are based on the sequential use of rate-zonal centrifugation and isopycnic banding centrifugation. The former depends on sedimentation rate (s) in a liquid density gradient, while the latter depends on equilibrium banding (p), also in a gradient. These have been combined in so-called s-p centrifugation. The unique finding is that in s-p plots, most viruses fall in an otherwise almost vacant area termed the “virus window” (Anderson, N. G. et al, Separation of Subcellular Components and Viruses by Combined Rate- and Isopycnic Zonal Centrifugation. Nat. Cancer Inst. Mongr. 21: 253-283, 1966). While a prototype system for making such separations was developed, no complete biologically-contained s-p system for making such separations safely when infectious pathogens are present has been developed, or is currently available.

The s-p system provides a means for recovering virus concentrates from large volumes of starting material. One version of this system employs continuous-sample-flow-with-banding (CSFWB) to achieve a separation based on sedimentation rate and banding density in one pass through the rotor. Centrifuges of this type have been used for large-scale vaccine purification, and the protocols developed are for the concentration of single viral species. Centrifuges of this type may be used directly for recovery of virus from plasma and large fluid volumes, and may also be used for low-speed prefractionation of tissue homogenates by removing cellular debris before a second high-speed centrifugation to concentrate the virus. Isolation of viruses by filtration is well known. Wallis et al, Annual Review of Microbiology 33:413-37 (1979).

In a previous invention (U.S. Pat. No. 6,254,834) applicants proposed to isolate viruses and virus-like particles using purely physical methods, as a means of providing new diagnostic tests, new drug targets and new knowledge of infectious agents.

Work on the so called “virion window” initially suggested that few particles existed in nature having the sedimentation rates and banding densities of viruses that were not viruses. Careful examination of phlegm (mucus), however, showed that it often contains particles within this range as observed by laser light scattering. Mucus is synthesized by a sequence of biochemical steps yielding aggregates of very large size. The glycoprotein building blocks are composed of several protein chains two-thirds covered by carbohydrate size chains attached by O-glycosidic linkages. The presence of a large amount of carbohydrate accounts for the high physical density of these particles. The individual protein chains are linked together by disulfide bridges and can be digested with trypsin. Treatment with reducing agents or trypsin breaks the larger aggregates into approximately 500 kDa structural units that are too small to interfere with virus isolation.

No general methods have been previously developed to separate and concentrate a range of different virus (or other infectious particles) away from contaminants that may be present initially that also yields them in a highly concentrated form, with all procedures done in contained, remotely-operated and controlled systems.

### SUMMARY OF THE INVENTION

The purpose of the present invention is to detect, isolate and characterize large numbers of infectious agents, including known and unknown agents, present in a mixture. All steps in the process, (except, optionally, the initial sample acquisition), may be carried out under remote control in biological containment.

A use of the present invention is to determine which known viruses or other infectious agents are presently circulating in a particular population by monitoring the occurrence and spread of infectious particles using pooled samples from that population.

A further use of the invention is to discover new previously undiscovered infectious agents, many of which are not culturable by any currently knows means.

A further purpose of the present invention is to rapidly identify and characterize any virus without culturing it and/or to rapidly identify and characterize any virus without the use of any virus-specific reagent or procedure. Another object of the present invention is to simultaneously identify plural different viruses in a mixed virus sample. This is preferably done exhaustively to identify all viruses in a mixed sample of many viruses, both known and previously unknown.

Another use of the present invention is to concentrate particles exhibiting sedimentation coefficients and isopycnic banding densities in the range characteristic of known infectious particles, to isolate the nucleic acids present in these particles, and sequence them to identify the source of their nucleic acids. An objective of the present discovery plan is to determine whether non-viral nucleic acid contaminants are present in the s-p samples.

An additional variation on the invention is to remove glycoproteins, such as mucus, from mixed samples by treatment of the concentrate with O-glycosidases, reducing agents, or proteases.

It is still another objective of the present invention to determine whether a biological material is virus-free.

It is yet another objective of the present invention to use continuous sample flow with banding centrifugation to concentrate infectious particles from a mixture of samples that may contain a mixture of plural infectious particles.

In the present invention one may shotgun sequence mixed preparations of virus nucleic acids obtained from virus concentrates produced by any means, including extraction from filters used to remove viruses from commercial human blood products.

It is another objective of the present invention to use antibody (IgG) preparations from pooled human or animal sera or antibody containing fractions, (e.g. gamma globulin) to isolate those viruses to which the hosts have been exposed from those to which the hosts have not been exposed. This technique restricts the viruses isolated to those, which bind antibodies in the antibody preparation employed, and reduces the number of extraneous viruses captured. The antibodies may be of human or animal origin. These approaches help to narrow down the focus to viruses of specific medical, agricultural or environmental importance.

The present invention achieves these results by first isolating a mixture of many viruses from a biological sample, or mixture of many biological samples, followed by separating mixed nucleic acids from the viruses, optionally fragmenting the nucleic acids and/or amplifying the nucleic acids by cloning or using the polymerase chain reaction. Sequencing the nucleic acids, searching viral genome databases to determine which sequences are from known viruses or from new previously unknown viruses, follows this. Thus, multiple known and/or unknown viruses may be simultaneously characterized The method may also be employed in quality control studies where demonstration of the absence of viruses is essential. If evidence for a virus or other infectious particle is discovered, the species and strain may be determined from the partial or complete nucleotide sequences obtained.

Different nucleic acid extraction and modification methods may be used to separate RNA and DNA. Nothing need be known about the virus before its isolation and characterization.

The present invention isolates virus particles using physical methods, extracts nucleic acids and then systematically sequences the nucleic acids and/or characterizes their proteins with the aim of developing and producing new diagnostic tests, new drug targets and new knowledge of infectious particles. For known viruses, the PCR may be used for amplification, while unknown viruses may be amplified by cloning. The present invention is applicable to pooled serum samples accumulating in clinical chemistry laboratories, and usually discarded. Such samples are representative of diseased local populations, include many with known infectious diseases, suspected infectious diseases, and diseases not currently associated with infectious agents. Such populations serve the function of sentinel populations, are, in several senses, random, and are continuously available. An overall objective of this invention is to provide a continuously operational means for determining “what is going around” either in the population generally, or in a defined population. This requires, the assembly of general means for obtaining representative samples. The samples may include pooled serum samples drawn assembled from hospitals, clinical laboratories, or other sources, sewage treatment plants, natural waters, human tissue samples removed at surgery or during biopsy, bandages, and all other body fluids including urine, feces tears, synovial fluid, CSF etc.

A preferred method for practicing this invention is a completely contained, remotely operated system for receiving putative agent-bearing samples and processing them all the way from initial samples to nucleic acid fragments ready for cloning or sequencing. These fragments are preferably automatically expelled from the system in a suitable labeled container, and contain no infectious agents. The system may or may not have gloves or other penetrable devices, be completely sealed during operation, and be completely sterilized internally at intervals. If so desired, sterility may be maintained by having the inner walls and equipment heated to a lethal temperature, e.g. 120° C. and have certain sample handling compartments be cooled as needed. For convenience in referencing it is termed a P-6 containment system.

### BRIEF DESCRIPTION OF THE DRAWINGS

FIG. 1 is a flow chart of the primary steps involved in the methodology of the present invention.

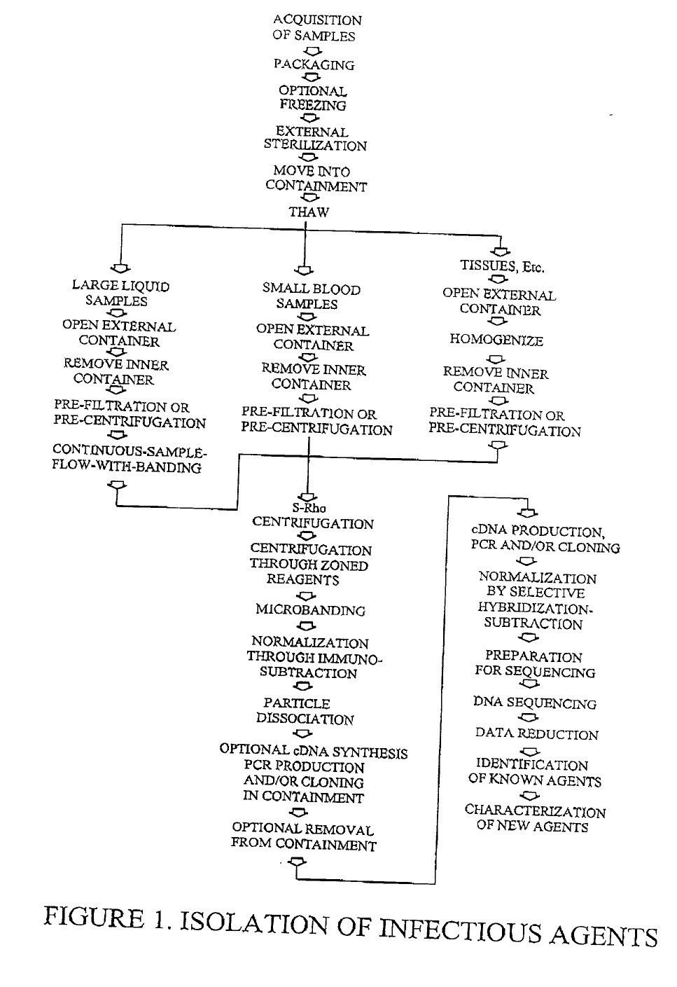

FIG. 2 depicts a contained system for performing the steps involving infectious materials.

### DESCRIPTION OF THE PREFERRED EMBODIMENTS

In this invention, methods of obtaining and characterizing unknown viruses are presented. These methods allow a more comprehensive search to be made than is possible with those previously described.

The term virus has been used for convenience in the text, however it is meant to be synonymous with the term “infectious particle” and is meant to embraces all conventional viruses and similar nucleic acid-containing particles including viroids, plasmids, nanobacteria, virus-like bacteria, and conventional microorganisms such as bacteria and fungi. If the microorganism is free-living and not necessarily parasitic, it need not actually be able to infect a host to be considered within this broadened definition (e.g., a non-infectious non-disease causing bacteria naturally found in the environment).

The term “new infectious particle” is an infectious particle having a nucleic acid sequence differing from any previously described by at least one nucleotide.

The term “antibiotic” refers to anti-viral, antibacterial or compounds or compositions that are inhibitory to the functioning or replication of an infectious agent.

The term “isolated”, when referring to a virus, means a composition that is essentially biologically free of other viruses or infectious particles. The term “purified” refers to a state where the relative concentration of a virus or other agent is significantly higher than in a composition where the virus is not purified.

The term “biological sample” includes tissues, fluids, solids, extracts and fractions that contain viruses or other infectious particles. These samples may be from an organism or from the environment.

The term “trait” includes both desirable and undesirable features of an organism. Inherited diseases or predisposition to diseases may be considered an undesirable trait. Traits may be due to genetic differences or by infection. The term “infectious particle” encompasses a whole infectious particle whether active or inactivated, one or more immunogenic proteins or peptides derived from the infectious particle (whether killed, attenuated or natural), or one or more compounds which elicits a specific immune response to said infectious particle or is recognized, usually by binding, by a past immune response to an infectious particle antigen. The antigen may be made synthetically or by a recombinant biological system and may be as small as a single epitope on a larger molecule.

The term “antibody” is meant to be broader than the traditional naturally occurring antibody but rather covers antisera, monoclonal antibodies, reassortant antibodies, recombinant microorganism (e.g. phage) display binding compounds, synthetic protein binding partners, Fab2, Fab, other fragments of any of these and any other specific protein binding molecules. The antibodies (if not synthetic) may be made from any species and in any species (not necessarily the same species) or culture.

The term “individual” encompasses any single animal, plant, and microorganism or human or subspecies collection thereof such as a strain or variety. An individual may be part of an individual organism, particularly when different parts are genetically distinct such as naturally occurring mutant sports commonly found on only one branch of a plant, etc.

The term “unculturable” refers to microorganisms and similar entities that do not replicate in culture or replicate so poorly, so slowly or with such great difficulty as to not be timely, affordable or in sufficient quantities for prompt testing. For example, M. tuberculosis typically requires 6 weeks in culture to grow noticeable colonies. For a patient where prompt diagnosis and antibiotic testing is desired, such slow growth is unacceptable and practically is little better than not culturable. Thus, the inclusion of such microbes in the definition. A preferred embodiment of the present invention involves the isolation of microorganisms followed by determining the sequence of various parts of their nucleic acids in microquantities (typically picograms to femtograms) from concentrates of particles having the physical properties of known infectious particles. The general process involves three main steps.

Initially, virus-enriched material from human, plant, animal and environmental sources to supplement the use of centrifugal separations applied to blood, serum, or tissue homogenates (as previously described). Additionally, one may use conventional filtration techniques and commercial products that are now used to remove virus particles from blood-derived pharmaceutical products. While the intent of these products is to remove a potentially dangerous contaminant from a pharmaceutical, the used filters retain the trapped virus (and other contaminants) as an enriched source of virus. Since these filters are typically used to filter large amounts of human serum and serum-derived protein products, prepared from large volumes of pooled human serum or plasma, it is likely that a wide variety of human blood-borne viruses are present in such a “filter-cake”. The material recovered from such filters can be further processed by one or two-dimensional centrifugal separations (as described below) to further enrich the viruses and remove non-viral material. The final concentration may be done by microbanding to yield a concentrated suspension in a few microliters of a gradient (See U.S. Pat. No. 6,254,834). The suspension may then be diluted to reduce the concentration of gradient solute, and resedimented in microbanding tube. Either with or without this further enrichment, the viral mixture can be disassembled (by releasing the viral nucleic acid and removal of the viral protein) and the sequence of the nucleic acids determined. The mixture of viral nucleic acids can be optionally cleaved, cloned or otherwise amplified for sequencing. The whole pool of viruses can be “shotgun” sequenced without separation of virus types, and the discrete viral genomes re-assembled on the basis of overlapping identical or complementary sequences similar as very large sections of whole chromosomes have been assembled from multiple short fragments.

Alternatively, if one suspects the nucleic acid sequence of a particular known infectious particle, a fragment of this nucleic may be hybridized to a single oligonucleotide or an array of oligonucleotide probes as a method for detecting the particular infectious particle's nucleic acid. In such a format, measuring exact complementary matches performs sequencing.

The present invention solves the technical challenge to viral particle isolation by getting rid of the up to ten million-fold excess of non-viral material, and by concentrating the virus in a high state of purity in a very small volume—all without a specific purification technique involving a known feature of the virus. The masses of individual virions may range from approximately 6×10⁻¹⁵ to 1.2×10⁻¹⁷ grams. In a tissue with 10⁸ cells per gram, and 10 infectious particles per cell, the mass of an virus, with a mass of 10⁻¹⁶ grams per virion, would be 10⁻⁷ grams per gram of tissue, while the total number of particles present would be 10⁹. The present invention addresses the problems of purification by physical methods without relying on any feature of any particular virus and thus is suitable for discovering new viruses.

When screening for microorganisms infecting a population, it is easier to use pooled samples to reduce the number of analyses necessary. Furthermore, it is easier to use a technique for assaying for multiplicity different microorganisms simultaneously. The present invention provides a method for simultaneous isolation of microorganisms of radically different classes, including all types of viruses and bacteria.

Biological samples may be pretreated with detergents, proteolytic, glycolytic, or lipolytic enzymes to remove contaminating material and release infectious particles from being bound. Pressure changes, sheering and other physical and/or chemical techniques may be employed to lyse host cells and disperse solid matter. Non-enveloped viruses are generally resistant to detergents. Many viruses and bacteria are resistant to many hydrolytic enzymes. This is particularly true of those that persist in the environment or are transmitted by the oral-fecal route.

Many viruses are selectively sensitive to proteases, lipases, detergents and organic solvents. For example, sedimentation of viruses through a gradient zone containing trypsin or other proteases under conditions where the exposure to the enzyme is brief will digest and change the sedimentation properties of many cytoplasmic contaminants. Similarly, brief exposure to low concentrations of non-ionic detergents will similarly disaggregate many cytoplasmic membranes. However, the most useful components of these zones are nucleases that will digest DNA and RNA, but leave nucleic acids enclosed in protein capsids or lipid-containing membranes intact. Thus viral concentrates may be sedimented through a zone containing 5 ug of trypsin/ml and/or 5 ug/ml of each of DNase and RNase. This zone may be immobilized at a density of approximately 1.04 g/ml above a gradient extending from 1.05 to 1.3 g/ml for Iodixanol™, with the virus sample overlayed at a density of 1.02. Native mucus solubilizes spontaneously but slowly. The first unit size observed in solution is about 15×10⁶ Da, but continues to break down further. In the present invention, nasal washings may be included in the samples processed. It appears probable that large quantities of virus are shed in mucus during the early stages of upper respiratory infections. As such infections are very common and are caused by a variety of microorganisms, such a sample material is particularly desired. Therefore, means and methods for depolymerizing mucus may be included in the concentration and isolation process.

When density gradient separations are included in the isolation procedure, detergents and enzymes can be imprisoned at specific levels in the gradients through which viruses are sedimented. Nucleases may be included among these enzymes to destroy any free nuclei acid present.

Sample preparation may typically involve homogenization and or dilution of solid tissue as well as liquid samples. Typical steps involve first grinding up fresh, frozen, or lyophilized tissue and suspending it in liquid. For example, human brain tissues from individuals who had active mental illness at the time of death from suicide are potential sources of new viruses. One may also correlate the presence of viral genes or genomes with the tissues being examined. This may suggest the location of action and give insights as to the cause of or aggregating factor in the corresponding disease for that tissue. In either situation, large cells and particles may be first removed by filtration, centrifugation or sedimentation before concentrating using the techniques of the present invention.

Preferred biological samples include blood, urine, feces, biological waste disposal from a clinical lab, air filter on a building/public location, sewage, waste fluids (feces, urine, blood) from an animal slaughterhouse, wastewater from agricultural and food processing plants, food or other plant material (e.g. cotton, oils, lumber) processing facility and samples of animals and plants in the natural environment. Experimentally produced and manufactured samples are included, for example, vaccine lots for quality assurance.

Typically mixed biological samples have many different types of infectious particles, some of interest and some simply incidentally present. Due to samples having very low concentrations of infectious particles, a specific method of enrichment is preferred, for example separating human infecting viruses from a mixture containing other viruses (including bacteriophage). A major application of this approach is the isolation of human pathogens from the very heterogeneous mixture of viruses (including animal and bacterial viruses) to be found in liquids from oceans, lakes, rivers, swimming pools, or sewage facilities.

An approach of a preferred embodiment is to introduce an additional enrichment step using human serum antibodies. In particular, human gamma globulin (a pool of IgG antibodies obtained from numerous human volunteers and sold commercially by many organizations including the Red Cross) is a known therapeutic protective against a broad range of human diseases (including viruses) because it contains antibodies made by numerous individuals who have been exposed to and mounted a successful antibody defense against numerous viruses.

These antibodies recognize and bind tightly to the viruses to which the donor was immune, and this property allows the mixture of antibodies in the gamma globulin product to recognize a broad array of human pathogens, known and unknown. These approaches help to eliminate extraneous viruses, to obtain rare pathogens and to narrow down the search of human viruses of possible medical importance.

The present invention may either 1) immobilize large amounts of the gamma globulin on a solid support (e.g. magnetic beads), allow the viruses to bind to the antibodies, wash out the unbound material (including non-human pathogens), and finally elute the human pathogenic viruses; or else 2) incubate the crude virus mixture with the antibody, separate the viruses from unbound antibody using density gradient centrifugation (the viruses, with or without bound antibody, are denser than the antibody protein), recover the virus band and expose this to a support capable of binding immunoglobulin (e.g., a protein A, protein G or anti-human Ig antibody covered surface), elute the unbound virus (i.e., that carrying no bound IgG and hence not recognized by the gamma globulin preparation). The virus is then determinable by recovering nucleic acid from the bound viruses and determining its sequence. In both cases, enriched infectious particles or the final nucleic acid preparations are recovered using a commercial gamma globulin therapeutic that “recognizes” a broad range of known and unknown human-infective viruses. Purpose-made mixtures of antibodies from convalescent individuals or individuals of having viral infections, or immunized individuals may also be used. Such collections could be made all over the world in order to ensure the presence in the immunoglobulin pool of antibodies against as wide a variety of human pathogens as possible, or as desirable.

In the present invention, methods for concentrating viruses from a large volume may be used. The starting biological sample may be any of a large number of infectious particle containing samples, alone or mixed with other samples also. Together, the combined operational system of the present invention may be used for searching tissue (sputum, feces, solid tissue (biopsy, resection or cadaver) and fluid samples (e.g. saliva, nasal washings, blood, urine, CSF, sweat, serum, plasma etc.), homogenates, agricultural product processing wastes, sewage, veterinary, slaughterhouse waste water, food processing facilities, various plants or plant parts combined or from different environmental sources (animal droppings, soil near trees, on rocks, where no plants are...), natural or contaminated waters for infectious particles. Further, the entire concentration process, as applied to potentially lethal infectious particles, may be performed robotically and/or in containment. Thus, the present invention is adaptable to a search relatively large numbers of samples for potentially harmful infectious particles.

Pooled samples from a particular country or region of the world or even from extraterrestrial sources (meteors, Martian, upper atmosphere samples, etc.) may be used to determine viruses presently circulating in certain populations or regions and which are exogenous. The present invention may be used to establish the existence of extraterrestrial life forms. Certain areas for high probability of finding new microorganisms may be monitored. For example, swine and/or duck workers or their animals may be periodically checked for new influenza viruses. Prostitutes may be monitored for new strains or antibiotic resistant sexually transmitted diseases. Pap smears, diagnostic laboratory samples and waste, blood banks, public toilets, air in subways, elevators and other public places with poor air flow may also be periodically checked for new infectious particles. Epidemiological surveys and monitoring for newly emerging diseases from any source may also be performed using the present invention.

Another embodiment of the present invention is to concentrate particles exhibiting sedimentation coefficients and isopycnic banding densities in the range characteristic of known infectious particles, and to isolate the DNA (if present) and/or to prepare cDNA from RNA that may be present. To further highlight the location of the specific desired band, detectable marker particle(s) may be added to the sample for easy identification and removal of the fraction likely to containing the desired infectious particles. Each detectable marker particle has a predefined density or a pre defined sedimentation coefficient or both. Initial concentration may be done by centrifugal or filtration techniques to remove the bulk of the sample from the infectious particle containing fraction. Large density marker beads have been described; density markers suitable for use in microbanding tubes are described here.

The behavior of many biological particles in density gradients may be manipulated by changing gradient conditions. These changes in properties may be exploited in the separations of the present invention. For example, different particles, including viruses, differ in their permeability to materials used to construct gradients, and may vary greatly in banding density depending on the salts used. Thus, two different particles may band together under one set of conditions, but band at very different density levels under others. As an example, DNA is very dense in CsCl while a conventional protein is much lighter. This behavior is reversed in some particles in the iodinated gradient materials such as Iodixanol®. Infectious particles as a group usually contain proteins and nucleic acids, and may contain lipids. Particles of non-infectious agent origin, which may occur in samples being processed, generally do not contain nucleic acids, and do not respond in the same way to changes in gradient composition as infectious particles. Further, non-infectious contaminants are generally very much more susceptible to digestion with proteases or nuclease, or to dissolution with mild detergents, than are intact infectious particles the preferred methods for concentrating viruses into very small volumes are the microbanding techniques exemplified in (U.S. Pat. No. 6,254,834). In this method, a gradient is formed which bands viruses in a very thin part of a tube such that very small quantities of virus are visualized by light scattering or fluorescence when the virus was stained by nucleic acid stains such as YOYO-1.

Zonal centrifuges, including those adapted to continuous-sample-flow-with-banding (CSFWB) have been developed to fractionate large quantities of complex mixtures based on sedimentation rate and banding density. The large-scale K series centrifuges in the CSFWB mode use a liquid density gradient held in place against the wall of a large cylindrical rotor to band virus sedimented out of a centripetal stream of virus-containing solution. Up to 150 liters of an influenza vaccine has been passed through such a rotor at 40,000 rpm in an eight-hour day, banding all of the virus into a zone approximately 150 ml in volume. More slowly sedimenting particles pass through the rotor while more rapidly sedimenting and/or denser particles sediment into and band in the narrow gradient held by centrifugal force against the rotor wall. The zone is recovered by first reorienting the gradient by slow deceleration, and then recovering the gradient at rest simply by draining the gradient out the bottom of the rotor. Another aspect of this invention is the detection of both DNA and RNA viruses simultaneously by first preparing cDNA by synthesis from the RNA and reverse transcriptase. Once the nucleic acids are extracted, any RNA present is converted into cDNA and then treated in the same manner as nucleic acid from DNA viruses. Should the extraction procedures affect one of the nucleic acids differentially, the sample may first be separated into two aliquots and a DNA extraction performed on one aliquot and a RNA extraction performed on the other aliquot in preparation for the synthesis of cDNA. This variation has the added advantage of distinguishing RNA viruses from DNA viruses.

After the nucleic acids have been extracted, their sequence is determined by a number of conventional methods. If one suspects the nature of the infectious particle, one may determine the sequence by PCR amplification and detection using specific primers and/or an oligonucleotide probe or series of oligonucleotides. Alternatively, the PCT amplification itself may be used as an assay as an amplification resistance test because a specific set of primers that don't amplify a nucleic acid is indicative of the lack of a nucleic acid containing sequences complementary to the primers in close proximity. This is particularly useful for typing the strain of influenza virus, determining the genotype and likely chemosensitivity of HIV, HCV, M. tuberculosis or other microorganisms. For infectious particles where less is known or suspected, sequencing after amplification is preferred. Amplification is traditionally performed by PCR with known or random primers or by ligation into a vector and cloning although other methods may also be performed. So-called “shotgun” cloning and sequencing is preferred.

The shotgun cloning of purified viruses may be performed by random shearing or cleavage with plural restriction enzymes followed by sequencing. By comparing overlapping fragments, entire viral sequences may be determined. Because sequences from different viruses usually will not be overlapping, a large number of different viruses may be detected and sequenced simultaneously. Additionally, viruses and different microorganisms (or nucleic acid fragments or plasmids or phage) may simultaneously be detected. While the present methodology has been optimized for mixtures of viruses, the same techniques apply to any infectious particle obtained in sufficient quantity.

The success of so called “shotgun” cloning in sequencing very large DNA sequences such as those of entire organism genomes or chromosomes suggests sequencing of relatively small nucleic acid genomes of viruses and other infectious particles is comparatively simple. In the present invention, physical methods are initially used for isolating small amounts of intact agents from infected samples, essentially free of host nucleic acids. These isolated nucleic acids from the infectious particles are amplified, or cloned, sequenced, and the clones ordered by identification of overlaps, thus sequencing the entire sequence of many different viruses, or the partial or complete sequences of more than one bacterium, simultaneously. As this technology has previously been applied to mixtures of 23 different chromosomes for sequencing human DNA, it is in principle, applicable to mixtures of many infectious particles, especially viruses. Since the length of the nucleic acid in a human chromosome is a few orders of magnitude greater than that of a small infectious particle, the present invention can theoretically detect hundreds or even many thousands of different infectious particles simultaneously.

When sequencing, the entire sequence need not be determined. Provided that a relatively unique sequence is determined, one may be able to identify at least some of the infectious particle(s) present by sequences a small fraction of all of the nucleic acids present. This is essentially the same principle as determining a gene by a sequence tag.

Having cloned at least part of the infectious particle's genome, it is then practical to insert any open reading frames in an expression vector and express the protein or portion of a protein so encoded. While not every expression vector will function, routine trial and error experimentation to obtain at least a low level of expression is within the abilities of the skilled artisan. The expressed protein is then usable as an antigen to detect convalescent antibodies, to elicit antibodies in vitro or in an animal system, for use as a diagnostic control, or to prepare a vaccine for preventative or therapeutic purposes. By choosing an appropriate fragment, fragments or an entire protein (by overlapping sequences to reconstruct the whole gene) suitable expression products (infectious particle antigen(s)) may be prepared to produce products to prevent, treat or detect the native infectious particle. Particularly preferred expression systems are those with human cell lines.

Viral genes from new and/or unculturable viruses may be used to produce specific viral proteins in vitro. These in turn may be used as antigens in clinical immunological tests, or may be used to prepare specific vaccines. The vaccines may contain single or multiple viral proteins or fragments of them. The viral genes may be transferred to plants for protein-gene-product production. The infectious particle antigen(s) may be used to immunize an animal (or cells therefrom for in-vitro immunization) to produce antibody to the antigen. Alternatively, antibody to the antigen may be obtained from recombinant microorganisms that express an antibody or similar binding partner encoded by a heterologous gene(s) on their surface. By immobilizing the infectious particle antigen(s) on a solid phase, an immunosorbent may be formed for binding convalescent or mixed antibody (e.g. gamma globulin) added thereto. The antibody is then eluted there from under conditions that disassociate antibodies form antigens. Both the antigen and the antibodies may be used diagnostically without modification or after conjugating to a label in any of a large number of well know immunoassay formats such as sandwich, competitive binding assays.

When sequences from a novel virus are obtained, and where these sequences are identified in clones, the amplified using conventional cloning, the clone may be used to produce viral gene products suitable for antibody production. These may be polyclonal, monoclonal or recombinant. The antibodies may be immobilized to isolate the corresponding infectious agents from concentrates, and to produce reagents to detect growth of the agent in tissue culture or in animals when no cytopathic effect is observed, or where there is no evidence for an infectious diseases. In this way replication and transmittal of an otherwise unculturable infectious agent may be detected. When an infectious particle is obtained in a relatively pure state and in sufficient quantity it may also be identified by mass spectrometry of proteins or peptides derived therefrom, or by the DNA, RNA or cDNA or restriction enzyme fragment mapping.

However, many problems prevent such techniques from being used outside the present invention. When several different viruses may be present, mixed answers will result. When the infectious particles constitute only a small fraction of the mass of the sample, background measurements overwhelm the desired measurement. When extraneous RNA or DNA is present, a systematic search for new infectious agents cannot be performed, as one cannot distinguish between viral and extraneous nucleic acids.

Another use and variation on the present invention is to isolate specific clonal human antibodies against specific infectious particles from the pooled antibodies present in the gamma globulin product. Using isolated infectious particles prepared by recovery from an infected source such as patient serum or by growing the virus in tissue culture, or purified viral coat proteins made in vitro by any of a variety of other means, one can prepare an immunoaffinity support (such as the commercially-available Poros™ or agarose bead supports) bearing covalently-immobilized infectious particles or their outer proteins. Large quantities of pooled human antibody can be passed over such a support, and the very small fraction of antibody that binds to the support will be comprised of antibodies against the infectious particle(s). These antibodies can be eluted from the support (e.g., using a buffer at pH 2.5), exchanged into a suitable buffer, and used directly or cleaved with the proteolytic enzyme papain (either in solution, or by passage over a column of immobilized papain) to yield Fab antibody fragments. Other cleavage methods to produce other antibody fragments may be used.

These fragments can be separated and resolved on a 2-D gel by conventional 2-D electrophoresis of the usual type used to resolve complex protein mixtures. For example, Anderson et al, Analytical Biochemistry 85:331-40 and 341-354 (1978), Anderson et al PNAS 74:5421-5 (1977) and Anderson et al, Electrophoresis 12:883-906 (1991). Preferably, no disulfide reduction reagent is used which would cause dissociation of the heavy and light chain portions of the Fab fragment. At sufficiently high gel resolution, a protein stain will reveal a series of protein spots at molecular weights around 45-50 kd, and exhibiting a broad distribution of isoelectric points. One or more of these spots can be excised and identified. A conventional protein spot identification technique is to cleave it with trypsin (or any of a variety of other proteolytic enzymes) and the resulting peptides recovered and subjected to mass spectrometry. MS can reveal the entire peptide sequence of both chains of the Fab molecule selected, and this sequence information can then be used to prepare a single-chain antibody (scFv) which can be produced in plants (or by various other means) to serve as a passive immunotherapy for treatment of infection with the originally selected virus. These techniques are known per se such as U.S. Pat. Nos. 4,816,249 and 5,866,785. It will be apparent to those skilled in the art that a variety of antibody-like protein constructs could be made based on the sequence derived by this procedure from the anti-viral human antibody.

Should the antibody be of non-human origin, the constant sequence may have it's amino acid sequence altered to resemble human antibody as has been done in other commercial therapeutic antibodies. For example, see U.S. Pat. No. 5,968,511.

An alternative method to obtain purified human antibody against a particular infectious particle is to separate the virus from blood or similar biological sample where the infectious particle already has antibody molecules bound thereto, regardless of their effectiveness. By microbanding infectious particles and recovering the desired fraction, antibody is stripped from the infectious particle by low pH (e.g. citrate buffer pH 2.5) or similar methods well known to break antibody/antigen binding. The antibody is then readily separated by filtration, centrifugation, ammonium sulfate precipitation etc. and may be used directly for diagnostic or therapeutic purposes. When used as a diagnostic, it is preferred to label it or use a secondary labeled antibody for easy detection. Using such a technique, a diagnostic assay may be prepared without even knowing the microorganism responsible for a disease. Alternatively, the antibody may be immobilized and used to recover an infectious particle, even if previously unknown.

Laboratory-acquired infections continue to be reported suggesting that conventional isolation and handling methods are not completely safe. For new and very highly infectious agents, a complete barrier system (classified as P-4) is currently used. P-4 systems are usually embodied in a series of barriers and culminate in the use of a full sealed and ventilated suit. Lesser levels of containment utilize sealed chambers with rubber or plastic gloves. These systems are inconvenient, are rarely used on a daily basis in routine operations, and are all subject to punctures. The present invention may be reduced to a series of routine operations that can be performed in a completely contained robotic system. In such a system a sample is introduced in a sealed container, which has been or is externally disinfected. The sample is opened remotely, and all operations performed robotically under operator control. Only cloned separated nucleic acid fragments are reintroduced into the laboratory environment.

The containment system is preferably completely enclosed with samples going through an air lock and sterilized waste products exiting the system. Inside the containment are a number of devices for performing each of the method steps. The system is robotically manipulated to avoid the need for gloves and the like. External to the system is a computer controller which automatically operates the robotic apparatus (liquid sample handling, infectious particle separation and extraction of nucleic acids from the infectious particles and preferably every other manipulable apparatus) in response to the specific type of sample added or is separately adjusted by an operator during at least part of the operation. If desired a camera may be mounted inside the system for visualization when one wishes to avoid a window. The controller communicates to the robot through a signal, preferably electrical, optical, radio frequency etc. such that any breech in the containment system is minimized. This has significant advantages over a glove box by not having certain seals, breakable or penetrable barriers etc. The output of the system is packaged nucleic acids, or fragments thereof, and waste that are preferably sterilized before leaving the system.

For a total containment system, the basic stages in practicing this embodiment of the invention are first, preparation of samples in suitable sealable, externally sterilizable, readily transportable containers, with machine-readable labels, which containers can be handled, opened, their contents removed by robotic means, and readily sterilized and disposed of. These samples may be stored for prolonged periods at low temperatures.

The second stage encompasses the preparation of each sample for fractionation. This may include pooling of samples, dilution of samples, or homogenization of samples.

The third general stage may be divided into a plurality of substeps, but includes those steps required to isolated particles within a defined range of sedimentation coefficients and banding densities. The exact specification of this range depends on the solutions used, their physical properties, and their temperatures.

The fourth general stage is concerned with the further differential purification of virions or other infectious particles, and separation from any non-nucleic acid containing contaminants that may be present.

The fifth stage relates to methods for extracting DNA, RNA or both from virus suspensions, simultaneous inactivation of all viruses, and separation of the nucleic acids from all proteins and other contaminants that might be present.

The sixth stage concerns processing the separated nucleic acids to a form that can be encapsulated and removed from the containment for cloning and sequencing, PCR amplification. Optionally this stage may include processing all the way to insertion of samples in sequencers.

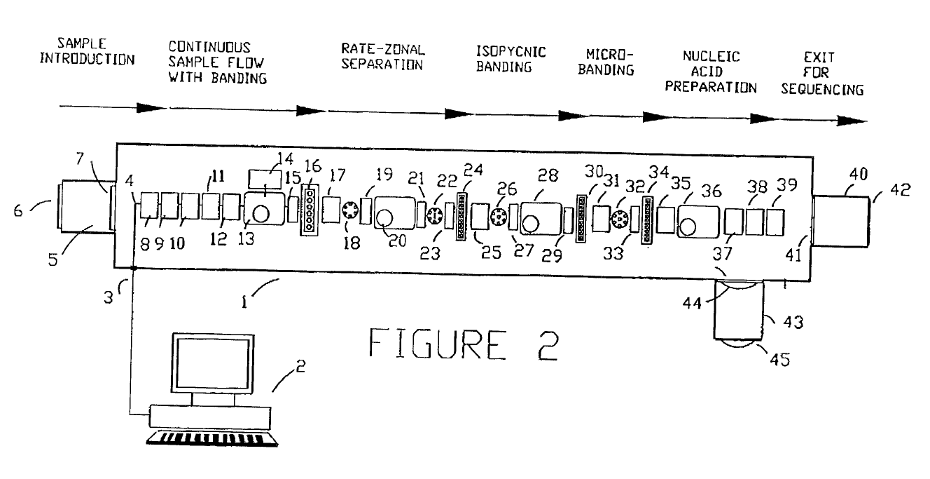

FIG. 2 illustrates diagrammatically the contained infectious agent system. Shown are the containment system 1, the computer controlling the entire system 2 through electrical or optical lines entering the containment through sealed connecting port 3 and continuing on inside 4 to connect to all the operational elements inside the containment, including door locks and sterilization systems (not shown). This system illustrates the isolation of viruses from pooled serum or plasma samples, but the principles apply to any large scale sample preparation, and with the addition of homogenization devices, to tissue samples as well. The sample entry port 5 through interlocked doors 6 and 7, allow samples to be introduced into the system. Port 5 may optionally incorporate sterilization of the external sample container.

Separate devices are diagrammatically illustrated, and are all automated and externally controlled. The operation of the system may be observed through transparent windows, but are preferentially monitored through TV cameras. Puncturable gloves are used in prototypes but are preferably avoided in the final operational system.

Automated devices in order include robotic systems to: open exterior containers 8, remove interior multiple containers 9, identify and pool samples 10, store pooled samples at low temperature 11, adjust sample composition including density and pH in 12, and feed sample into continuous-sample flow-with-with banding centrifuge 13, with gradient solutions provided by 14 and band recovery solutions also provided by 14. The recovered gradient is monitored by UV absorbance monitor and physical density monitor 15, the fractions are collected in response to density measurements in tubes 16. These in turn are introduced by 17 into tubes and the tubes loaded into swinging bucket rotor 18, which in turn is moved by 19 into centrifuge 20. After high speed rate-zonal separation centrifugation is complete, the rotor 22 (18) is removed from centrifuge 20 by device 21, and unloaded with UV monitoring by device 23 to yield a series of fraction comprising different fractions 24 containing particles separated on the basis of sedimentation rates. The fractions 24 are transferred by device 25 into rotor 26 which may be either an angle head rotor or a swinging bucket rotor to separate particles on the basis of their isopycnic banding densities. Rotor 26 is transferred by device 27 to centrifuge 28 for isopycnic banding after which the rotor is removed by device 29 that scans the tubes and recovered bands into a new series of tubes 30. These fractions are transferred, with diluting solutions, by device 31 into high speed microbanding tubes and further into rotor 32 which may be spun in centrifuge 28 or a similar centrifuge added at this point in the series. Tubes are removed from rotor 32 by device 32 and constitute the fractions in rack 34. These fractions are recovered by device 35, inactivation solutions added, and centrifuges in low speed centrifuge 36. The recovered fractions are unloaded in device 37, further fractionated into loaded into small bar-coded tubes for DNA sequencing. The exterior surfaces of these tubes and rack are disinfected tin device 38, and stored in preparation for sequencing in device 39. Samples for sequencing are removed through port 40 through interlocked doors 41 and 42. Waste materials are removed through port 43, which may comprise an autoclave sealed through interior doors 44 and exterior doors 45.

Many versions of this system are within the limits of this description, the sequence of devices may be changed, and additional ones added as those skilled in the arts use it experimentally.

It should be noted that the Human Genome Project, and other large sequencing projects, have developed very large excess DNA sequencing capacity, and that faster and larger sequencers will continue to be developed. Further, the successful development of so-called shotgun sequencing to the human genome means that tens of thousands of different viruses can, in theory, be simultaneously sequenced.

In any population study certain viruses will predominate, and very rare, and possibly more important, trace viruses may be missed. Therefore means are required for removing these predominant viruses during the fractionation procedures outlined. For example, certain coliphages predominate in rivers and streams contaminated with human or animal sewage. Systematic application of the system and process described here will reveal these, and antibodies prepared for each of them. Such antibodies may then be used (e.g. immobilized on suitable supports) to remove these predominant viruses from concentrated suspensions. (Note that a similar process may be used to isolate trace viruses that are being sought.) These processes may be used to normalize a virus suspension.

Concentrated viral suspensions prepared from pooled samples obtained from a relatively large local population have many uses. With the application of large scale cloning, estimates of the frequency of occurrence of different viral diseases may be obtained by counting the number of times sequences from one virus are found. This allows epidemics to be detected before the causal agents can be determined by conventional means. Using immobilized DNA microarrays, in which sequences from known viruses are immobilized; it will be possible to determine more rapidly which viral genomes are present in a mixture. In addition, quantitative PCR may be more effectively applied, and economically applied to the detection of larger numbers of different viruses in a centralized facility. In addition, random primers may amplify DNA where the exact primers are unknown.

Methods for producing cDNA from viral DNA are well known, but have not generally been applied to the systematic analysis of mixtures of viral RNA and DNA. Digestion of genomic DNA and cDNA with at least two different restriction enzymes to produce overlapping sequences, and the assembly of overlapping sequences to reconstitute viral genomes is a well-known general cloning technique.

Hence, given viral concentrates from large numbers of patient samples, the problem of finding causal agents is two-fold. First the agents must be concentrated from relatively large volumes, and second; contaminating human genomic DNA must be removed as far as possible, and, in any case, recognized. Given current human genome data, there is little difficulty in identifying contaminating human DNA. However if there is much of it, and if it is carried through to sequencing, then a large fraction of the sequencing effort may be wasted.

Hence the inclusion of gradient processes for sedimenting putative viral particles through density gradient layers containing DNase may be used, and have been developed below. Most DNA, however, can be removed by either rate-zonal centrifugation to leave the DNA behind during sedimentation, or by isopycnic zonal centrifugation in cesium chloride or similar dense ionic medium where the DNA is much heavier than known viruses, and hence bands at a higher density.

The problems of building an integrated contained remotely controlled system including very high-speed centrifugation that can be repeatedly and effectively sterilized is within the province of available technology given the teachings of this specification.

By determining the presence, absence, increase or decrease in the abundance of specific pathogens, the initial stages of an epidemic may be detected. In addition, when a new antiviral or antibiotic compound is to be tested in human subjects, it is of great advantage to know it its early stages when an outbreak of the susceptible agent(s) begins.

Electron microscopy has long been used to count and characterize infectious agents. However there has been no systemic attempt to combine morphological data provided by EM with banding density data to characterize unknown agents as may be used in the present invention.

Infectious agents, except prions, characteristically contain nucleic acids, which may be stained with fluorescent stains (e.g. TOTO, YOYO, etc. dyes) that become intensely fluorescent when bound to DNA or RNA. Some of these stains bind differently to different types of nucleic acid and may be used not only for detection but also for characterization. They have not previously been used to follow infectious agents during purification, or to characterize them during gradient separations. Given the completion of sequencing of the human and other genomes it is evident that identification of contaminating host DNA or RNA is a relatively straightforward process.

The systems and procedures described in the present invention are therefore useful in epidemiological studies, in drug development studies, and can serve as the tripwire of the outbreak of bioterrorist or biological warfare agents. The present invention is not limited to discovery of new agents but may be used for quality control of all microbial production systems. Additionally, quality control of any pharmaceutical or biological may be checked for contamination by searching for unculturable microorganisms. For example, research products such as fetal calf serum and food or feed products intended for consumption may also be screened for the presence of known or unknown infectious particles using the techniques of the present invention.

The same techniques may be used to check for culturable microorganisms as well. In the field of gene therapy using replacement genes in replication-defective viral particles, the present invention provides a method for quantification and detection of revertant or replication competent virus as well as titering replication defective viral particles.

It is another use of the present invention to determine infectious particle contamination in a material. If one is concerned with host material contamination such as blood cells in plasma or serum products or conversely viral contamination of a cell culture, the present invention is sufficiently generic to detect either. This is particularly a problem when preparing attenuated vaccines, replicative defective viral particles, and the like. A small contamination with wild type virus or other microorganism can be disastrous. The method of the present invention may rapidly identify such contaminants, optionally by amplification with primers to amplify the region(s) differing between wild type and mutant.

Pathogenic microorganisms of cellular form, bacteria, fungi, parasites are themselves preyed upon by viruses. Bacteriophage have been used clinically to treat bacterial infection and are still so used in certain countries. By discovering new version of such viruses, one may find even more viruses that may be of therapeutic use to treat bacterial, fungal and parasitic infections. The discovery of new viruses, which infect pathogens by the methods of the present invention, represents possibilities for new therapeutics for those pathogens and is part of the present invention.

In addition to shotgun sequencing of the viral nucleic acids, one can take mixtures of the proteins released from pools of mixed infectious particles and separate and analyze the proteins by conventional biochemical separation techniques or proteomics technology. For example, a 2-D gel separation of the mixed viral particle proteins reveals a large number of protein spots varying in abundance in accordance with the number of copies of the respective virus in the pool and with the number of copies of the particular protein in each viral particle. These proteins can be further identified (e.g. by mass spectrometry) and the resulting data compared to the nucleic acid sequences recovered from this and other viral sequence databases to identify the virus genome that codes for each protein. This then allows one to know which viral genes code for proteins found in the viral particle (thus identifying them as candidate antigens for diagnostics and antiviral vaccine therapy), and it allows us to establish a rough quantitative estimate of the virus's abundance in the pool (based on the relative abundance of its protein subunits). Thus, infectious particle identification by nucleic acid sequence is indirect by simply characterizing the proteins. This method may be used simultaneously or in conjunction with nucleic acid analysis.

New strains of some microorganisms that differ by as little as one nucleotide may be detected. A one-nucleotide change may indicate host susceptibility or antibiotic agent susceptibility or diagnostic detectability.

As some variation and mutation in the sequence of many viruses exists, one can determine a map of polymorphisms and mutations for a given virus that will be particularly helpful in preparing vaccines, determining pathogenicity etc. Influenza in particular is constantly changing its sequence and thus prompts monitoring and rapid identification of new strains is an important use for the present invention. Likewise, subtyping viral strains and discovering new strains, such as HPV strains in cervical cell samples, may be performed with the present invention. This method may be used to distinguish high-risk oncogenic HPV strains from non-oncogenic strains occurring in a population. Likewise, the method may distinguish between interferon responsive and resistant strains of hepatitis C virus and other sequence differences in various other microorganisms, regardless of whether the sequences are coding sequences. While exemplified with viruses, the method is equally applicable to other infectious particles.

One of the problems facing modem disease detection is to finding out which microorganism goes with which disease, if indeed any do. Many of these microbes may produce effects only evident long after the initial infection. For example, if schizophrenia is a late effect of a virus, as has been proposed, then the virus may not be present when the disease is evident. Convalescent antibodies may be present however. By cloning and expressing one or more proteins coded for by the microorganism, detection of convalescent antibodies is possible and thus associating a microbe with a disease even in the absence of the microbe itself using the methods of the present invention. When a new putative infectious agent is discovered, large numbers of sera from normal and diseased individuals may be analyzed using micro-versions of the technology described here or other conventional immunoassays to discover associations between specific nucleic acid sequences for unculturable agents and specific diseases.

This approach, if successful, will result in a specific diagnostic assay for identifying infections as they occur, providing appropriate antibiotic therapy and for producing new vaccines to prevent or treat them or new diagnostic antigens. Further, if a new agent is discovered, and a source of the active infectious agent can be found, then it may be possible to survey a wide variety of cells in culture, media or species to find one or more that would support growth. This in turn could return that agent to the normal, and current, culture-related technologies of conventional clinical microbiology and virology. Different strains of microorganisms, plants and animals are believed to result from genetic differences. However, when observed phenotypically, it is not readily apparent whether the difference is caused by universal infection or inherited viruses. It is believed that many different strains are actually the same basic organism with or without differing viruses infecting them. For example, Corynebacterium diphtheria by itself is a harmless bacteria living in the human throat. However, when the bacteria are infected by a particular bacteriophage, the bacteria produce a toxin causing a deadly disease. Without knowledge of the bacteriophage, one might assume them to be two different strains or even two different species. With the present invention, one now has the ability to determine the virus or other infectious particle cause for some strain differences. Such knowledge is of use for genetic engineering and plant and animal breeding, and in the treatment of human diseases.

Diseases that tend to occur in families of plants and animals have been assumed to result from inherited genetic mutations or environmental effects including taught lifestyle and behavior. Other diseases such as schizophrenia, Alzheimer's, heart attacks, etc. were thought to be the results of other causes even when they tend to occur in families. Distinguishing traits that are not strictly diseases are likewise to be from the same causes. Many multifactorial conditions and the concepts of genetic penetrance and expressivity contain the assumption that at least one unknown factor is responsible for the phenotype not matching the genotype. “Inherited” diseases previously thought to be genetic have often been shown to be otherwise. HTLV-1 is a virus that often passes from mother to children. Certain cancers caused by HTLV-1 appear to be inherited but are actually of viral origin. It is likely that other diseases that tend to run in families actually endogenous viruses or viral diseases of low transmissibility. The same is applicable to animals and plants as well that share a similar environment as their relatives. The diseases caused by viroids represented a great unknown for many years as scientists lacked the tools for finding the cause. Even today, the tools for systematic discovery of infectious agents are insensitive and imprecise. The present invention provides one with the ability to find such viruses and may be used for screening biological samples from individuals for the establishment of a viral cause of the disease or trait.

With greater knowledge of genomics, the concept of disease vs. trait has blurred. Within the genome of many microorganisms, plants and animals, many pseudogenes and proviral sequences are integrated. The effects of these are unclear but maybe as significant as a mutation in a normal structural gene. Depending on the location of a proviral sequence, transposons and the like, the biological effect is the same as or even more dramatic that of a mutation. These sequences may also encode one or more proteins, which also may have a phenotypic effect, especially if they should be incorporated into an extrachromosomal particle.

With the techniques of the present invention, one finally has the ability to identify all viruses, proviruses, other infectious particles, and other nucleic acid-carrying particles, regardless of their culturability and our lack of knowledge about them. This allows one to make many new associations between the virus causing a particular trait or the removal of an infectious agent or a nucleic acid bearing particle from a host cell imparting a different trait.

Determination of virus-free animal products is of importance for international transport of companion animals, livestock, meat, and byproducts such as fetal calf serum etc. Such a determination may yield a higher price. By using the present invention one may determine whether a product is virus free, including free of unknown viruses.

Likewise, persistent infection of plants with a virus is a common cause for slow growth or low produce yields. The sale of “virus-free” plants is an established business and the determination of being virus free is an important function. Before the present invention, one was limited to detecting known viruses. Given the present invention, finding unknown viruses with a variety of desirable and undesirable effects on the plant will provide new tools to the plant breeder for selecting certain traits.

Even with bacteria, only a few percent of environmental bacteria are culturable in spite of great efforts over many years to culture more. As microorganisms produce many of today's antibiotics and other pharmaceutical compounds, identification and characterization of additional microbes is desirable and may be performed by the present invention. Given the nucleic acid sequence of the microorganism, proteins may be expressed and pharmacological properties assayed. Additionally, enzymatic activity displayed by the unculturable microorganism may also be of commercial value. Using the same general technique, new enzymes may be expressed even when the host microorganism is not culturable and does not produce detectable amounts of the enzyme.

For example, relatively few microorganisms living in high temperatures (hot springs, geysers, volcanoes, ocean vents, etc.) can be cultured. However, finding additional thermostable enzymes is particularly desired for DNA polymerase, proteases, lipases, amylases etc. By sequencing at least a portion of the unculturable infectious particles from those environmental sources, one can predict which open reading frames may code for desired enzymes based on sequence similarity to known microbe enzymes. The gene is then synthesized (or removed from the mixed nucleic acids and then be inserted into an expression vector) and expressed to produce the enzyme. At no point is the infectious particle cultured.

Another advantage of the present invention is that one can isolate viruses before or while an individual or a group exhibits symptoms that bring a new outbreak to the attention of the health community. This is of great advantage in defense of biological warfare agents, malicious vandalism, criminal activity, etc. as a threat can be distinguished from actual danger. During a natural outbreak of certain diseases (e.g. Ebola, anthrax, etc.), once symptoms appear, it is generally too late for treatment of those initially infected. Likewise, antibiotic treatment for other diseases (e.g. cholera) is much more effective if given prophylactically. In still other diseases (e.g. tuberculosis), the time spent for culturing the suspected pathogen is too long before treatment should begin. In the situation of a carrier, it would be desirable to detect carrier status directly rather than by indirect means such as antisera titer, or by tracing illness to individual carriers. In all of these situations, the nonspecific rapid viral or microbial detection system of the present invention may be used. The present invention is further described by reference to the following examples, which are offered by way of illustration and are not intended to limit the invention in any manner. Many standard techniques well known in the art or the techniques specifically described below were utilized.

The invention now will be exemplified in the following non-limiting examples.

### EXAMPLE 1

Viral Isolation from Tissues

A mixture of the following viruses was added to a 10% homogenate of unperfused Fisher 144 adult male rat liver prepared in 0.25 M sucrose: 1. Lambda phage (dsDNA) 1.5×10¹⁰ plaque forming units; 2. M13 phage (ssDNA) 1.0×10¹⁰ pfu; MS2 phage (ssRNA) 3×10¹⁰ pfu; and Phi 6 (dsRNA) 1.36×10⁹ pfu. The viruses were suspended in phosphate buffered saline at pH 7.2. 20 ml of the homogenate was layered over a 10-40% w/w sucrose gradient in a Beckman Ti-15 titanium rotor spinning at 3,000 rpm at 5° C. The speed was increased to 30,000 rpm and after 30 minutes the rotor was unloaded and the gradient collected in 15 ml fractions. The tubes representing fractions sedimenting between 80 S and 1,500 S and at a density between 1.05 and 1.3 gm/ml were collected, and were placed in 30 ml centrifuge tubes, 20 g of dry CsCl added to each, small aliquots of fluorescent density beads (densities 1.074, 1.114, 1.188, and 1.373 g/ml) were added and the tubes spun overnight at 28,000 rpm at 20° C., during which time the CsCl went into solution and formed steep gradients. These were aligned in a special illumination apparatus and photographed to reveal the bands. In some experiments the fluorescent DNA-binding dye TOTO-1 was added to assist in visualized the viruses. The fluorescent bands were recovered using long gel-sample layering pipette tips and a pipetter attached to a fine vertical movement. The nucleic acids from individual bands may be isolated and treated as described below. Alternatively, the entire volume containing particles in the sedimentation range from 80 S to 1,500 S may be recovered, and banding in a smaller Ti-14 rotor over CsCl, and the portion of the gradient having the density range for banding viruses collected. This fraction may be either pelleted for nucleic acid extraction, or banded again in a smaller gradient, followed by banding in a microbanding tube. All the banding procedures may be followed using density beads. The final banding volume may be as little as 10 microliters.

### EXAMPLE 2

Infectious Particles from Large Volumes

For large fluid volumes, the initial concentration is being done in a K-II continuous-flow-with-banding centrifuge (Anderson et al, Analytical Biochemistry 32:460-494 (1969)) and the portion of the gradient known to contain viruses recovered from the gradient after centrifugation. As much as 100 liters of sample may be processed per day with such machines. If the gradient decays during the CSFWB procedure, additional volumes of the solution may be introduced to the dense end of the gradient. The recovered viral band, usually 200-300 ml in volume, is further concentrated either by pelleting, by isopycnic banding in a Z-14 rotor, followed by further banding in tubes as described above. If a Z-14 rotor is used, the solutions are preferably diluted between isopycnic banding steps. Using the further steps outlined in Example 1, the viral zones are further concentrated in preparation for sequencing.

### EXAMPLE 3

Infectious Particles from Dilute Liquids

For relatively clear starting suspensions, for example sea or river water, or plasma, initial clarification is done by slow speed centrifugation or filtration. The clarified suspension is then subjected to ultrafiltration with a size exclusion to remove and concentrate virus particles. These viruses are recovered from the pharmaceutical ultrafilters by reverse flow and further concentrated for sequencing as described above.

### EXAMPLE 4

Isolation and Sequencing of Viral Nucleic Acids

Following preparation an enriched virus fraction from the microbanded viruses present in the sample, concentrated virus samples are subjected to nucleic acid extractions using a standard buffer (50 mM Tris-HCl, pH 8.0, containing 0.5% SDS, 20 mM EDTA and 3 mg Bentonite clay/ml) and two successive phenol:chloroform:isoamyl alcohol (25:24:1) extractions. The aqueous phase is retained during both extractions and one tenth volume of 3 M sodium acetate pH 5.4 is added and 2.5 volumes of absolute ethanol for total nucleic acid (RNA and DNA) precipitation. The mixture is incubated in −80° C. for 30 min, brought to room temperature and then centrifuged at 4° C. for 30 min. Is washed twice with 70% ethanol and the nucleic acid pellet resuspended in TE buffer. Total nucleic acid content is determined using micro-spectrometric techniques measuring absorbance at 260 and 280 wavelengths.

The viral nucleic acids may be composed of a single virus species or a population of species representing viruses with single stranded RNA, double stranded RNA, single stranded DNA and double stranded DNA genomes. To capture the information from all members of a population the sample is split into three aliquots to obtain data from: 1) double stranded DNA, 2) single stranded DNA and 3) single and double stranded RNA. As the distribution and type of viruses vary with the sample, flexibility in the cloning process and the use of multiple different techniques may be necessary. These are within the abilities of the skilled artisan given the description of the present invention. Reactions are carried out to clone comprehensive samples of the genomes of the organisms and then genome fragments subjected to DNA sequencing. The fragments are then assembled using standard bioinformatics approaches described below, e.g. programs PHRED, PHRAP. CLUSTAL and CONSED. The reassembled genomes or genome fragments are BLAST analyzed with publicly available sequences of DNA clones and ESTs to establish the identity of the virus(s) of interest in the sample. Open reading frame predictions may be derived and protein family motif searches can be used to augment the construction of the virus phylogeny and relationship trees. This data is then used to detect the identity of the virus(es) in the sample, and further investigation into diagnostic and therapeutic applications.

Double stranded (ds) DNA samples may be quite large (e.g. Poxvirus) or small (e.g. polyomavirus), but nevertheless suffer from shearing effects of isolation. To seek to restore sheared ends and loose the minimal amount of genetic information, the DNA is incubated with T4 DNA polymerase using 100 mcM of each dNTP, 1-3 units of T4 DNA polymerase (NEB, Beverley, Mass.) per mcg of DNA in standard buffer (refer to NEB catalog). This sample is incubated at 12° C. for 20 minutes. This reaction will fill-in lacking bases in the case of a 5′ overhang to form a blunted end and will remove excess 3′ overhang nucleotides to form a blunted end via the enzymes 3′ to 5′ exonuclease activity. Following the incubation, the reaction is incubated at 75° C. for 10 minutes to inactivate the enzyme. The prepared DNA sample is then digested with 2-4 restriction endonucleases that recognize 4 base sites. Examples of “4-base cutters” include: Sau3AI (GATC), AluI (AGCT), TaqI (TCGA), DpnI (GATC), NlaIII (CATG) and others, which can be obtained through NEB. The number of enzymes chosen such that the size of a random piece of DNA incubated with the mixture of enzymes would be cleaved into an average of 500 bp fragments. Enzymes can be chosen to favor AT or GC rich DNA content if desired. Following digestion, the DNA fragments are ligated into standard cloning vectors (such as pUC 19, pBluescript, or others). Aliquots of the digest are ligated into vectors digested with BamHI (Sau3AI digested DNA), SphI (NlaIII digested DNA), ClaI (TaqI digested DNA) or SmaI (AluI or DpnI digested DNA) and treated with calf-alkaline phosphatase to prevent vector self-ligation. The aliquots allow a different population of restriction fragments to be ligated into different vectors based on restriction site compatibility. This approach yields a population of fragments that are completely digested and contain no internal enzyme recognition sites for the 4 base cutters or incompletely digested and contain internal recognition sites for one or more 4 base cutters. The proportion of each is dependent on the amount of enzyme in a digestion reaction, the length of incubation time and the content of the DNA surrounding individual recognition sites. If DNA amounts are limiting, restriction enzymes yielding blunt DNA ends may be used and oligonucleotides can be ligated to the ends. PCR amplification can then be used to increase the amount of DNA for downstream processes, but will also loose the normalization (equal representation) of the virus genome fragments.

DNA ligations are transformed into competent E. coli cells (such as DH5a) and plated onto agarose containing ampicillin selectable antibiotic (plasmids contain the bla gene that encodes a gene product rendering E. coli insensitive to the action of this antibiotic). At this point, 94 colonies will be picked and two control plasmids. Cultures are grown at 37° C. for 20 hours and then glycerol stocks and DNA preparation are made. DNA is digested with two restriction endonucleases that recognize sites flanking the insertion site of the vector. These diagnostic DNA digests are analyzed by electrophoresis in agarose gels and ethidium bromide staining. Insertions in plasmids are scored based on the liberation of a DNA fragment between 50 or greater bp in the digest. If greater than 80% of the colonies contain an insert, more colonies will be picked.

The largest common dsDNA viruses are members of the poxviridae with genomes of ˜300,000 bp. 3-4 fold coverage of the genome would need to be accomplished if a complete reconstitution of the genome is desired, or single fold coverage if a diagnostic outcome is sufficient. Therefore, between 600 (1×) and 2400 (4×) colonies would need to be picked, DNA prepared and subjected to standard automated sequencing approaches. Automated robots are available for such purposes. Universal primers are used for M13 forward and M13 reverse sites in the vectors or conversely, primers recognizing T7, T3 or SP6 phage promoters may be used. The issue of choosing primers that will give sequence of the insertion in both directions is to be optimized for each sample and requires routine trial and error experimentation and is within the abilities of the skilled artisan. ABI 377, ABI 3700 or Amersham Megabase sequencers can be used along with their preferred chemistries. The result of this analysis is between 1200 and 4800 sequencing reactions that should give largely complete coverage of a 300 kb genome.

The DNA sample from single stranded (ss) DNA viruses can come from a variety of virus families which have ssDNA as a replication intermediate or packaged form of their genome, including parvovirus, circoviruses, and retroviruses among others. In order to capture the genetic information from these viruses, one must convert the ssDNA into a dsDNA form. This is most efficiently done by using T4 DNA polymerase or the Klenow fragment of E. coli DNA polymerase I to polymerize complementary DNA fragments from a population of random primers annealed to the virus nucleic acid. The products of the random priming reactions are a set of medium to small sized dsDNA fragments. Representation of the ends of the virus genome may be incomplete due to the constraints from the approach. PCR amplification can be used to increase the amount of DNA for downstream processes, but will also loose the normalization (equal representation) of the virus genome fragments.

The prepared dsDNA sample is then treated as above. The size range for viruses with ssDNA intermediates or packaged genomes range from ˜3 kb (circoviruses) to ˜12 kb (retroviruses). 3-4 fold coverage of the genome would need to be accomplished if a complete reconstitution of the genome is desired, or single fold coverage if a diagnostic outcome is sufficient. Therefore, between 24 (1×) and 96 (4×) colonies would need to be picked, DNA prepared and subjected to standard automated sequencing approaches as described above. The result of this analysis would be between 48 and 192 sequencing reactions that should give largely complete coverage of a 12 kb genome.

The most common type of genome for human, animal and plant viruses is that built from RNA. Viruses with ssRNA (Togaviruses, picomaviruses, coronaviruses, rhabdoviruses, paramyxoviruses, arenaviruses, retroviruses etc.) and dsRNA genomes (reoviruses, rotaviruses) comprise diverse genome structures and replications strategies. The inherent physical properties of RNA-RNA hybrids necessitates the nucleic acid to be strongly denatured in order to allow access to ssRNA stretches for sequence cloning. Viral nucleic acids are denatured with heat and methyl mercury reagents to separate RNA secondary structures (ssRNA viruses) and RNA strands (dsRNA viruses). Random DNA primers are added into the annealing reaction to allow for subsequent reactions. Following denaturation, reverse transcriptase (MMLV, AMV, SuperScript II—low RNase H, or others) are added to the reaction and allowed to extend products for ˜1.5 hours. Following this reaction, terminal transferase reactions are carried out to tag the copy DNA (cDNA) with a poly C or G tail. This step will enhance the ability to obtain the full ends of the RT products. The RNA component of the reaction is then removed by nuclease digestion. The second strand of the cDNA is synthesized using a primer complementary to that of the terminal transferase “tail” (if the tail is poly C, a poly G primer is used, for example). The results of these reactions are a single or series of dsDNA cDNA samples.

The prepared cDNA sample will then be digested and sequenced as above.

With a large database of viruses from all virus families available and powerful sequence homology search programs, the identity of a virus sample should be readily determined. Alternatively, if the virus is “new”, this should also be established by the methodology of the present invention. In many cases, the entire set of DNA fragments required to biochemically reconstitute the intact genome would be determined. The reconstruction of the virus genome is readily accomplished by the above method with standard molecular biology techniques and patience. This method allows multiple viruses from different families or genome content to be equally cloned and characterized from the same mixed sample. The lack of PCR in this approach will keep bias low as one obtains information concerning the actual content of the viruses in the sample. This method effectively diagnoses the type; species of virus and possibly quantity of virus in a sample and bioinformatically identifies the DNA fragments encoding important viral genes for reconstitution and use in diagnostic, vaccine or therapeutic approaches.

### EXAMPLE 5

Sequencing and Comparing

The complete DNA sequence of a gene or genome is the ultimate physical map. However, it is useful to construct intermediate level physical maps from cloned fragments: These cloned fragments can subsequently be used for sequencing or other manipulations. A library of such clones can be compared to each other and those that overlap aligned and placed in position on the chromosome relative to each other. The sets of clones, called contigs or contiguated clones, can then be checked for stability, exact representation of the starting genome, etc.

For smaller sequencing projects, such as virus genomes, one may use plasmid vectors and perform shotgun sequencing. To make a shotgun library, genomic DNA is sheared or restricted to yield random fragments of the required size (usually about 1 kb). The fragments are cloned into a universal vector and sequencing reactions are performed with a universal primer on a random selection of the clones in the shotgun library. The library of sub-fragments is sampled at random, and a number of sequence reads generated (using a universal primer directing sequencing from within the cloning vector). These sequencing reads are assembled into contigs and identifying gaps. The gaps are then targeted for sequencing to produce the full sequenced molecule. As with large insert libraries, the representation of the clone in the sub-fragment library can be non-random. This results in gaps in the preliminary assembled sequence. Directed sequencing can fill these gaps with primers derived from known flanking sequence, or selection of sub-clones spanning the gap. One commonly used strategy is to make two (or more) different sub-clone libraries with different insert size averages (say <1 kb and 4-6 kb). End sequence is generated from both ends of a subset of clones from each library. If a gap is flanked by the left and right side reads from a clone, that clone will contain the DNA found in the gap, and can be selected for directed sequencing. For DNA with a biased base composition, or containing large arrays of near-identical repeats, alternate strategies using sub-clone libraries with very small (˜200 bp) inserts can permit effective sequencing.

In the assembly phase, all the sequence reads from the clone are first compared to each other. Identities between the sequences of different reads are noted, and these identities are used to align the sequences into sequence contigs. The sequences of two different reads of the same segment of DNA may not be identical because of the quality of the sequencing reaction analysis. Thus for each base in the contig it is usual to require that it is independently confirmed from multiple overlapping reads from both directions. Contig building software has been designed that takes into account the “quality” of each base in a read. The term quality is a measure of the confidence the software has that the base has been called correctly. Any gaps, discrepancies or ambiguities in the sequence can be flagged for re-sequencing, possibly using alternate chemistry.

Depending on the shotgun approach employed, it may include DNA preparation from clones, shearing/cloning of DNA (shotgun library construction), random sequencing reactions, primer walking and finishing reactions as required and contig assembly. A set of programs from the University of Washington; phred, phrap (Ewing, B. et al (1998) Genome Res. 8:175-185; Ewing, B. and Green, P. (1998) Genome Res. 8:186-194), and consed (Gordon, D. et al. (1998) Genome Res. 8:195-202) may be used to assemble the individual sequences into contiguous sequences. The chromatogram files are processed prior to assembly to identify high quality bases, trim off sequencing vector, and remove contaminating sequences as well as other options. There is a choice of various assembly programs. Phrap is especially useful when phred quality scores are available. The following steps are implemented using phrap:

1. Reads are compared pairwise using a fast implementation of the Smith-Waterman algorithm. Alignment scores are then adjusted to reflect the qualities of discrepant bases, and these adjusted scores rank the list of matches. At this stage anomalous reads (e.g. chimeras) are also identified.

2. A greedy assembly algorithm is used to construct a layout of read overlaps, based on the pairwise comparisons.

3. The contig sequence is constructed from the layout as a “mosaic” of the highest quality parts of the reads; this is done by finding an optimal path through an appropriately defined weighted directed graph.

4. The quality of the assembly is analyzed by enumerating discrepancies between reads and the contig sequence, “weak joins” that are potential sites of miss-assembly, and consistency of forward/reverse read pairs.

5. A probability of error (reflecting the amount and quality of trace data) is computed for each sequence position. This can be used to focus human editing on particular regions, and to automate decision-making about where additional data is needed.

6. Finally, the consed editor is used to view the contigs, bring up the traces for editing, design primers to fill gaps, and export the consensus sequences.

Once the primary sequence of an infectious agent has been determined, it is preferred to proceed to try and identify all the genes and genetic elements encoded in the sequence. Gene prediction is the identification of coding segments of a genome: these can be RNA genes or protein coding genes. For ribosomal RNA genes, identification is by sequence similarity to known ribosomal RNA genes. For tRNAs, simple sequence similarity search can be used to identify many genes, but more sophisticated statistical models may be used to find most tRNAs. The best models appear to be hidden Markov chain type models.

A suit of tools such as GeneMark (Borodovsky, M. and McIninch (1993) Computers Chem. 17: 123-133; Blattner, F. R. et al. (1993) Nucleic Acids Res. 21:5408-5417), BLAST (Altschul, S. F. et al. (1990) J. Mol. Biol. 215:403-410; Altschul, S. F. et al. (1997) Nucleic Acids Res. 25:3389-3402), FASTA (Pearson, W. R. and D. J. Lipman (1998) Proc. Natl. Acad. Sci. 85:2444-2448), and the PFAM (Krogh, A. et al. (1994) J. Mol. Biol., 235:1501-1531) database are used to aid in the annotation and open reading frame prediction phase of gene prediction. Also, for protein-coding genes several sets of evidence are used to lead to predictions of genes, including: comparison to cDNA sequence, derived from a mRNA in a sample: this confirms that a segment of DNA is transcribed. Presence of an open reading frame with no stop codons. Presence of the requisite start site consensus and terminator or poly adenylation signals. Presence of matches to splice site consensuses. Presence of a bias in base composition (in AT or GC-rich genomes, coding genes will stand out as regions of GC or AT richness, respectively). Presence of a bias in base frequency. This is usually assessed as the bias over three or six bases, and is linked to the codon phasing of the protein coding genes. It is also possible to reveal this by Fourier transform analysis: a significant signal is found at a spacing of 3 bases, and Ability to code for peptides with significant similarity to other known protein sequences.

For bacterial, viral, and yeast genomes, where there are no introns, a potential gene need only have a start codon, an open reading frame of some length, and a stop codon. Additional transcription initiation signals and terminators may be present. In a random DNA sequence of any length there is a finite possibility of getting long open reading frames by chance. The probability of getting short ORFs by chance is high, and thus most gene prediction programs do not accept genes less than a minimum number of residues (e.g. 50 amino acids).

### EXAMPLE 6

Detection and Generation of Virus-Free Germ Stock

Using the procedures described in Examples 1, 4 and 5, extracts of presumed infectious-agent free strawberry plants are prepared and processed to determine whether nucleic acids from infectious particles are present. The germ stock, if infected, is grown in the presence of antiviral agents, or is rendered agent-free on outgrowth by conventional means. The germ stock is then retested to prove that it is infectious-agent free.

### EXAMPLE 7

Characterization of Infectious Agents by Mass Spectrometry

Virus concentrates are prepared by the methods of Examples 1 and 4 and the proteins are separated by conventional two-dimensional electrophoresis. Protein spots are excised and examined by MALDI mass spectrometry to determine the masses of each protein including the capsid subunits. These masses may be compared with a database containing the measured or calculated masses of these subunits. In addition, the isolated infectious agent suspensions or the isolated protein spots are digested with trypsin or another proteolytic agent to produce peptides that are then characterized by mass spectrometry, and these masses compared with those actually measured using isolated peptides or calculated from sequence data. In addition, such peptides are partially or completely sequenced by LC-MS/MS mass spectrometry.

It will be understood that various modifications may be made to the embodiments disclosed herein. Therefore, the above description should not be construed as limiting, but merely as illustrations and exemplifications of preferred embodiments. Those skilled in the art will envision other modifications within the scope and spirit of the claims appended hereto.

All patents and references cited herein are explicitly incorporated by reference in their entirety.

What is claimed is:

1. A method for identifying a plurality of infectious particles in a sample comprising;
separating an infectious particle containing fraction,
extracting at least two nucleic acids from the fraction,
sequencing at least a portion of the at least two nucleic acids or a complementary sequence thereof, and
determining the identity of the infectious particles from the sequence or by overlapping sequences derived from plural nucleic acids.

2. The method of claim 1 wherein the sample is a mixture of biological samples from plural individuals.

3. The method of claim 1 further comprising comparing the sequence of the nucleic acids to a database of known sequences.

4. The method of claim 3 wherein a new infectious particle is detected.

5. The method of claim 4 wherein a known infectious particle is simultaneously detected.

6. The method of claim 4 wherein plural new infectious particles are simultaneously detected.

7. The method of claim 1 wherein the infectious particle is not cultured.

8. The method of claim 1 wherein the nucleic acids are amplified in copy number between extracting and sequencing.

9. The method of claim 1 wherein said fraction is separated by centrifugation.

10. The method of claim 9 wherein the infectious particles band at a density between 1.05 and 1.3 gm/ml and exhibit sedimentation coefficients between 80 and 1,500 S.

11. The method of claim 1 wherein said fraction is separated by filtration and a retentate is recovered.

12. The method of claim 1 wherein said at least one nucleic acid is RNA and further comprising synthesizing a DNA complementary to said RNA.

13. The method of claim 3 wherein the database contains nucleic acid sequences from known infectious particles or the sequences of the species from which the biological sample is obtained.

14. The method of claim 1 wherein said nucleic acids are cleaved such that overlapping fragments are formed.

15. The new infectious particle identified by the method of claim 4.

16. The method of claim 1 wherein the sample is an aliquot from a composition intended for contacting a living organism.

17. The composition tested by the method of claim 16 and the results indicate no unwanted infectious particles are present.

18. A true infectious particle-free germ stock which lacks detectable infectious particles having densities of 1.05 to 1.3 gm/ml and sedimentation coefficients of 80 to 1,500 S.

19. The true infectious particle-free germ stock of claim 18 wherein the infectious particles are dsDNA, ssDNA, ssRNA or dsRNA viruses.

20. An oligonucleotide of at least 13 nucleotides complementary to or the same as the nucleic acid sequence of said new infectious particle of claim 4 and which is not complementary to a nucleic acid sequence of a known infectious particle.

21. A method for producing antibody against an infectious particle comprising;
separating an infectious particle with antibody bound thereto containing fraction from a biological sample
contacting said infectious particle with antibody bound thereto with a reagent and under conditions which disassociates the antibody from the infectious particle, and
recovering free antibody.

22. The method of claim 21 wherein the separating is centrifuging a biological fluid.

23. The method of claim 22 wherein the fraction is a band at a density between 1.05 and 1.3 gm/ml and a sedimentation coefficient between 80 and 1,500 S,

24. The method of claim 21 wherein the biological fluid is a mixed biological fluid from multiple individuals.

25. The method of claim 21 wherein plural free antibodies to plural infectious particles are recovered.

26. Free antibody specific for an infectious particle produced by the process of claim 21.

27. The method of claim 21 further comprising directly or indirectly attaching said free antibody to a label or solid phase.

28. Free antibody specific for an infectious particle directly or indirectly attached to a label or a solid phase produced by the process of claim 27.

29. A method for separating infectious particle specific antibody comprising;
immobilizing an infectious particle antigen on a solid phase,
contacting the immobilized infectious particle antigen with a mixed antibody containing liquid from plural individuals for a time sufficient for specific antibody to be bound to immobilized infectious particle antigen, and
separating the solid phase containing the specific antibody from the liquid.

30. The method according to claim 29 further comprising eluting and recovering the specific antibody from the solid phase.

31. The method of claim 29 wherein the mixed antibody containing liquid is from a plurality of healthy individuals with no sign of current or past infection from the infectious particle.

32. A specific antibody to an infectious particle composition produced by the process of claim 30.

33. The method of claim 30 further comprising directly or indirectly attaching said antibody to a label or solid phase.

34. The antibody specific for an infectious particle directly or indirectly attached to a label or a solid phase produced by the process of claim 33.

35. A specific antibody to an infectious particle composition produced by the process of claim 31.

36. The method of claim 31 further comprising directly or indirectly attaching said antibody to a label or solid phase.

37. The antibody specific for an infectious particle directly or indirectly attached to a label or a solid phase produced by the process of claim 36.

38. A method for producing specific antibody to a new infectious particle comprising;
immobilizing an antigen of the new infectious particle identified by the method of claim 4 on a solid phase,
contacting the antigen with an antibody containing liquid from for a time sufficient for specific antibody to be bound to immobilized antigen, and
separating the solid phase containing the specific antibody from the liquid.

39. The method of claim 38 further comprising eluting and recovering the specific antibody.

40. The method of claim 38 wherein the antibody containing liquid is convalescent serum from at least one individual exposed to the new infectious particle.

41. The method of claim 38 wherein the antibody containing liquid is a mixed liquid from plural individuals with no sign of current or past infection from the infectious particle.

42. A specific antibody to a new infectious particle composition produced by the process of claim 39.

43. The method of claim 39 further comprising directly or indirectly attaching said free antibody to a label or solid phase.

44. The antibody specific for an infectious particle directly or indirectly attached to a label or a solid phase produced by the process of claim 43.

45. A method for making an antibody comprising;
determining at least a part of an amino acid sequence of the antibody of claim 26, 32, 35, or 42,
obtaining a DNA encoding at least a part of the determined amino acid sequence,
expressing the DNA to produce an expressed antibody having at least a part of the determined amino acid sequence, and
recovering the expressed antibody.

46. The expressed antibody produced by the method of claim 45.

47. A method for purifying an infectious particle antigen comprising;
immobilizing the antibody of claims 26, 32, 35 or 42 to a solid phase,
contacting an infectious particle antigen containing liquid with the solid phase for a time sufficient to allow immobilized antibody to bind to the new infectious particle, separating the solid phase from the liquid.

48. The method of claim 47 further comprising eluting and recovering the infectious particle antigen.

49. The infectious particle antigen produced by the method of claim 42.

50. The method of claim 42 further comprising directly or indirectly attaching said infectious particle antigen to a label or solid phase.

51. The infectious particle antigen directly or indirectly attached to a label or a solid phase produced by the process of claim 44.

52. A method for making an antigen comprising;
determining at least a part of an amino acid sequence of an antigen of the infectious particle of claim 15 or the antigen of claim 43,
obtaining a DNA encoding at least a part of the determined amino acid sequence,
expressing the DNA to produce an expressed antigen having at least a part of the determined amino acid sequence, and
recovering the expressed antigen.

53. The expressed antigen produced by the method of claim 46.

54. A vaccine comprising an infectious particle antigen of claim 47 and a pharmaceutically acceptable carrier.

55. A method for enriching a mixture of infectious particles in a biological sample for those particles that infect a host comprising;
obtaining antibodies from the host or a group of hosts;
contacting the biological sample with the antibodies; and
recovering infectious particles bound to the antibodies.

56. The method of claim 49 wherein the antibodies are obtained from multiple healthy people.

57. The method of claim 49 wherein the antibodies are obtained from one or more patients who have contracted or recovered from a known or suspected viral disease.

58. The method of claim 49 wherein the antibodies are obtained from individuals from multiple geographic, social, cultural, climatic or historical regions.

59. The method of claims 1, 21, 29, 38, 47 or 49 wherein the method is performed in a containment system.

60. An apparatus for manipulating infectious agents comprising;
a containment system having at least one air lock and enclosing devices for manipulating an infectious agent,
robotic apparatus for liquid sample handling inside the containment system,
robotic apparatus for infectious particle separation inside the containment system,
robotic apparatus for extracting nucleic acids from the infectious particles inside the containment system,
an adjustable controller outside the containment system, and
signal connections between the adjustable controller and each robotic apparatus inside the containment system,
wherein the controller effects operation of the robotic apparatus.

61. The apparatus of claim 54 wherein infectious agents enter but live infectious agents do not leave the containment system.

62. The apparatus of claim 54 wherein every controllable apparatus inside the containment system is controlled by the controller.
